SEQUENCE LISTING (25 sequences)

SEQ ID NO: 1 - protein, 4 aa, Artificial Sequence
  Q-peptide of parent construct sacrificial protein
MAGK

SEQ ID NO: 2 - protein, 3 aa, Artificial Sequence
  Q-peptide of parent construct sacrificial protein
VIR

SEQ ID NO: 3 - protein, 9 aa, Artificial Sequence
  Q-peptide of parent protein adenylate kinase
GFLIDGYPR

SEQ ID NO: 4 - protein, 15 aa, Artificial Sequence
  Q-peptide of parent protein triose phosphate isomerase
VVLAYEPVWAIGTGK

SEQ ID NO: 5 - protein, 10 aa, Artificial Sequence
  Q-peptide of parent protein apolipoprotein A1
NLAPYSDELR

SEQ ID NO: 6 - protein, 11 aa, Artificial Sequence
  Q-peptide of parent protein myosin binding protein C
GDQLFTATEGR

SEQ ID NO: 7 - protein, 16 aa, Artificial Sequence
  Q-peptide of parent protein alpha actin
SYELPDGQVITIGNER

SEQ ID NO: 8 - protein, 11 aa, Artificial Sequence
  Q-peptide of parent protein lactate dehydrogenase B
QVVESAYEVIR

SEQ ID NO: 9 - protein, 12 aa, Artificial Sequence
  Q-peptide of parent protein beta enolase
LITGEQLGEIYR

SEQ ID NO: 10 - protein, 13 aa, Artificial Sequence
  Q-peptide of parent protein alpha tropomyosin
ATDAESEVASLNR

SEQ ID NO: 11 - protein, 10 aa, Artificial Sequence
  Q-peptide of parent protein myosin heavy chain (embryonic)
SLEDQLSEIK

SEQ ID NO: 12 - protein, 12 aa, Artificial Sequence
  Q-peptide of parent protein glycogen phosphorylase
VLYPNDNFFEGK

SEQ ID NO: 13 - protein, 15 aa, Artificial Sequence
  Q-peptide of parent protein aldolase B
GILAADESVGTMGNR

SEQ ID NO: 14 - protein, 13 aa, Artificial Sequence
  Q-peptide of parent protein beta tropomyosin
ATDAEAEVASLNR

SEQ ID NO: 15 - protein, 14 aa, Artificial Sequence
  Q-peptide of parent protein myosin heavy chain (adult)
LQNEVEDLMVDVER

SEQ ID NO: 16 - protein, 14 aa, Artificial Sequence
  Q-peptide of parent protein glyceraldehyde 3-phosphate dehydrogenase
LVSWYDNEFGYSNR

SEQ ID NO: 17 - protein, 17 aa, Artificial Sequence
  Q-peptide of parent protein phosphoglycerate kinase
ALESPERPFLAILGGAK

SEQ ID NO: 18 - protein, 11 aa, Artificial Sequence
  Q-peptide of parent protein lactate dehydrogenase A
QVVDSAYEVIK

SEQ ID NO: 19 - protein, 18 aa, Artificial Sequence
  Q-peptide of parent protein alpha enolase
AAVPSGASTGIYEALELR

SEQ ID NO: 20 - protein, 18 aa, Artificial Sequence
  Q-peptide of parent protein actin polymerization inhibitor
LLPSESALLPAPGSPYGR

SEQ ID NO: 21 - protein, 16 aa, Artificial Sequence
  Q-peptide of parent protein pyruvate kinase
FGVEQNVDMVFASFIR

SEQ ID NO: 22 - protein, 21 aa, Artificial Sequence
  Q-peptide of parent protein creatine kinase
GTGGVDTAAVGAVFDISNADR

SEQ ID NO: 23 - protein, 3 aa, Artificial Sequence
  Q-peptide of parent construct sacrificial protein
AGK

SEQ ID NO: 24 - protein, 9 aa, Artificial Sequence
  Q-peptide of parent construct quantification protein
VICSAEGSK

SEQ ID NO: 25 - protein, 12 aa, Artificial Sequence
  Q-peptide of parent construct purification tag protein
LAAALEHHHHHH

(19) United States
(12) Patent Application Publication
(10) Pub. No.: US 2009/0137050 A1
(43) Pub. Date: May 28, 2009

(54) Artificial protein, method for absolute quantification of proteins and uses thereof

(75) Inventors: Julie M. Pratt, Leicester (GB); Robert Beynon, Merseyside (GB); Simon Gaskell, Altrincham (GB)

Correspondence Address: JAGTIANI + GUTTAG, 10363-A DEMOCRACY LANE, FAIRFAX, VA, 22030, US

(73) Assignee: PolyQuant GmbH, Regensburg (DE)

(21) Appl. No.: 11/916,292

(22) PCT Filed: Nov. 18, 2005
(86) PCT No.: PCT/EP05/12530

§ 371 Date: Aug. 11, 2008

(30) Foreign Application Priority Data
Jun. 2, 2005 (EP) 05090161.0

(51) Int. Cl. G01N 33/68; C07K 7/00; C07K 14/00; C40B 40/10; C12N 15/00

(52) U.S. Cl. 436/63; 530/328; 530/327; 530/326; 530/325; 530/324; 530/350; 506/17; 435/320.1

(57) ABSTRACT

The invention provides an artificial protein for quantitative analysis of the proteome of a sample, cell or organism, comprising at least two consecutive peptides linked by a cleavage sequence for separating the peptides; a singular marker on one or more peptide for determination of the absolute amount of this fragment; and N-terminal and C-terminal extensions for protection of the peptides; wherein each peptide represents one single protein of the sample, cell or organism and each peptide is in a defined stoichiometry. The invention further provides a collection of peptides, a vector and a kit comprising the artificial protein and a method for quantitative analysis of the proteome.

### FIELD OF THE INVENTION

The present invention relates to proteomics and more specifically to absolute quantification of proteins.

### BACKGROUND OF THE INVENTION

The need for absolute quantification in proteomics is becoming increasingly urgent. The most promising method is based on stable isotope dilution involving simultaneous determination of representative proteolytic peptides and stable isotope labeled analogs. The principal limitation to widespread implementation of this approach is the availability of standard signature peptides in accurately known amounts.

The two primary themes in proteomics are protein identification and the comparison of protein expression levels in two physiological or pathological states (comparative proteomics). The long term goal of being able to define the entire proteome of a cell is still unrealized, but the characterization of many thousands of proteins in a single analysis is now attainable.

For proteomics to become a platform technology serving the emergent field of systems biology, there is a pressing need for enhancement of quantification (Righetti, Eur J Mass Spectrom 10 (2004), 335-348). Most comparative proteomics studies deliver relative quantification, expressing the changes in amount of a protein in the context of a second cellular state (for example Dunkley, Mol Cell Proteomics (2004); Hoang, J Biomol Tech 14 (2003), 216-233; Ong, Mol Cell Proteomics (2002), 376-386).

However, the goal must ultimately be to define the cellular concentrations of proteins absolutely, whether as molarities or as numbers of molecules per cell. Absolute quantification, which poses one of the greatest challenges in proteomics, draws on well-established precepts in analytical chemistry, and requires either external standards or internal standards (Sechi, Curr Opin Chem Biol 7 (2003), 70-77; Julka, J Proteome Res 3 (2004), 350-363). External standardization is typified by immunodetection, whether solution phase or on position-addressable anti-body arrays (Walter, Trends Mol Med 8 (2002), 250-253; Lopez, J Chromatogr B Analyt Technol Biomed Life Sci 787 (2003), 19-27). The second approach, reliant on internal standardization, is based on mass spectrometry (MS), wherein highly selective detection of ions (or ion fragmentations) characteristic of the analytes of interest is combined with the use of internal standards.

In the most rigorous MS analyses, stable isotopic variants of the analytes are used as internal standards. The key underlying principle is that the determination of relative signal intensities during mass spectrometric analysis can be converted into absolute quantities of analyte by reference to an authentic standard available in known amounts. Direct application of this approach to intact proteins is impractical, and it is common to adopt the principle of surrogacy, that is to quantify indirectly by reference to a proteolytic peptide derived from the protein of interest.

Analyses based on these principles have been dubbed “AQUA” (absolute quantification) using internal standards synthesized de novo by chemical methods (Gerber, PNAS 100 (2003), 6940-6945). However, this approach does not lend itself well to absolute quantification of large numbers of proteins, as each Q-peptide would need to be chemically synthesised and independently quantified.

The international patent application PCT/US03/17686, published as WO 03/102220 provides methods to determine the absolute quantity of proteins pre-sent in a biological sample. The principle of WO 03/102220 is based on the generation of an ordered array of differentially isotopically tagged pairs of peptides, wherein each pair represents a unique protein, a specific protein isoform or a specifically modified form of a protein. One element of the peptide pairs is a synthetically generated, external standard and the other element of the pair is a peptide generated by enzymatic digestion of the proteins in a sample mixture. For performing the method of WO 03/102220 the standard peptides are calibrated so that absolute amounts are known and added for comparison and quantification. A sample of interest is also labelled with the same isotope tag as used for the standard peptides except differing in the isotopic label. The pairs of signals, which correspond to differentially labelled sample and standard peptides are finally observed and related to a list of expected masses based on the particular standard peptides included. The disadvantage of WO 03/102220 is that standard peptides need to be individually synthesised, purified and quantified. Moreover, both the sample and the standard peptides need to be specifically labelled separately, increasing the potential for variability between experiments.

Thus there is still an existing need to develop easy and convenient methods for absolute quantification in proteomics.

### SUMMARY OF THE INVENTION

The present invention is directed to an artificial protein for quantitative analysis of the proteome of a sample, cell or organism, comprising:
(a) at least two consecutive peptides linked by a cleavage sequence for separating the peptides;
(b) a singular marker on one or more peptides for determination of the absolute amount of the protein; and
(c) N-terminal and C-terminal extensions for protection of the peptides;
   wherein each peptide represents one single protein of the sample, cell or organism and each peptide is in a defined stoichiometry.

For the purpose of the invention the artificial protein is also named QCAT protein and the peptides used for the QCAT protein are called Q-peptides.

The cleavage sequence between two Q-peptides may be an enzymatic or a chemical cleavage sequence.

The N-terminal and C-terminal extensions protect the quantification peptides from processing and exoproteolysis. The artificial protein further includes features which allow for easy purification of the Q-CAT protein.

Each of the Q-peptides represents one single protein of the sample, cell or organism and each peptide is in a defined stoichiometry, which is typically, but not exclusively 1:1.

The present invention further concerns a collection of Q-peptides, which covers the complete proteome of an organism. This collection allows for rapid quantification of the proteome of such an organism.

The present invention also concerns a vector comprising the QCAT protein and a kit comprising the vector and/or the QCAT protein.

Moreover, the invention is directed to a method for quantitative analysis of the proteome of a sample, cell or organism, comprising the steps of:
(a) quantifying the amount of the protein or one peptide containing the singular marker in an absolute manner;
(b) generating a preparation of the proteins to be quantified;
(c) mixing the products of steps (a) and (b);
(d) completely cleaving the artificial protein of any of claims 1-10 and the proteins to be quantified in step (b) at the cleavage sequence;
(e) determining the quantitative amount of peptides;
(f) calculating the absolute amount of peptides,
wherein the artificial protein and/or the peptides are isotopically labelled.

It is important to note that the proteins do not have to be purified—partially purified followed by high resolution separation technologies would be equally acceptable.

### BRIEF DESCRIPTION OF THE DRAWINGS

FIG. 1 shows examples of Q-peptides selected as signature peptides;

FIG. 2 shows the DNA sequence, translated protein sequence and features of the QCAT;

FIG. 3 shows the Characterisation of the QCAT protein and Q-peptides;

FIG. 4 shows the Quantification using the QCAT protein, and

FIG. 5 shows the Use of the QCAT for muscle protein quantification.

### DETAILED DESCRIPTION OF THE INVENTION

The inventors describe here the design, expression and use of artificial proteins that are concatamers of tryptic Q-peptides for a series of proteins, generated by gene design de nova. The artificial protein, a concatamer of Q-peptides (“QCAT”) is designed to include both N-terminal and C-terminal extensions. The function of the extensions is to protect the true Q-peptides, to introduce a purification tag (such as a His-tag) and a sole cysteine residue for quantification of the QCAT.

The novel gene is inserted into a high-level expression vector and expressed in a heterologous expression system such as E. coli. Within the QCAT protein, each Q-peptide is in a defined stoichiometry (typically, but not exclusively 1:1), such that the entire set of concatenated Q-peptides can be quantified in molar terms by determination of the QCAT protein. Moreover, the QCAT protein is readily produced in unlabelled or labelled form by growth of the expression strain in defined medium containing the chosen label.

The inventors have successfully designed and constructed an artificial gene encoding a concatenation of tryptic peptides (a QCAT protein) from over 20 proteins. The protein further includes features for quantification and purification. The artificial protein was expressed in E. coli and synthesis of the correct product was proven by mass spectrometry. The QCAT protein is readily digested with trypsin; is easily quantified and can be used for absolute quantification of proteins. This strategy brings within reach the accurate and absolute quantification of large numbers of proteins in proteomics studies. Additionally, the QCAT was labelled by selective incorporation of a stable isotope labelled amino acid or by incorporation of ¹⁵N nitrogen atoms at every position in the protein.

It is therefore an object of the present invention to provide an artificial protein for quantitative analysis of the proteome of a sample, cell or organism, comprising:
(a) at least two consecutive peptides linked by a cleavage sequence for separating the peptides;
(b) a singular marker on one or more peptides for determination of the absolute amount of the protein; and
(c) N-terminal and C-terminal extensions for protection of the peptides;
   wherein each peptide represents one single protein of the sample, cell or organism and each peptide is in a defined stoichiometry.

In a preferred embodiment of the invention, the artificial protein comprises 10-200 peptides, preferably 10-100 peptides, more preferred 20-60 peptides, most preferred 60 peptides. It is possible to include multiple instances of specific peptides to modify the stoichiometry.

In yet another embodiment, the cleavage sequence is cleaved by a protease, preferably by trypsin and the singular marker is a cysteine residue.

Further, one or more peptides of the artificial protein are repeated identically at least one time, preferably one or more times, to achieve a particular stoichiometry between all peptides sequences.

It is preferred to include an affinity tag (e.g. a histidine tag) for purification of the protein. It is particularly preferred to include the affinity tag in either the N-terminal or C-terminal extensions of the protein.

In a further embodiment, the protein is labelled by an isotope, which is selected from the group consisting of ¹³C, ¹⁵N, ²H and ¹⁸O.

In yet a further embodiment each peptide comprises between about 3 and 40 amino acids, preferably about 15 amino acids.

The protein may comprise a molecular weight of about 10-300 kDa, preferably about 150-200 kDa, most preferred about 150 kDa and is preferably expressed in E. coli.

The origin of the proteome, i.e. the sample, cell or organism is preferably a mouse, rat, ape or human, but can be from any proteinaceous source.

In a preferred embodiment of the invention, the peptides represent different conformational, metabolic or modification states of the protein, in order to quantify all proteins derived posttranslationally from such a protein.

Further, the peptides of the artificial protein have preferably a defined molecular weight distribution and quantitative ratios. A mass spectrometer can be calibrated, preferably with molecular weights, which result in equidistant mass spectroscopy signals. Preferably, one or more peptides are represented twice in order to unambiguously label a reference molecular weight for calibration.

The invention is further directed to a collection of peptides, as defined in step (b) of claim 1, which covers the complete proteome of an organism. All expressed proteins of an organism are defined as the proteome of such organism. This allows for rapid quantification of the proteome of such organism.

Further included in the disclosure of the present invention is the absolute quantification of the protein levels of a certain reference strain, which can then be used to compare the protein levels of this particular strain or similar strains under varying experimental conditions.

The invention is also directed to a vector comprising a nucleic acid encoding the artificial protein and a kit comprising the artificial protein and/or the nucleic acid encoding the artificial protein. Further the invention is directed to a method for quantitative analysis of the proteome of an organism, which is described in detail above.

The present invention will be better understood by the encompassed examples and results with reference to the accompanying figures.

### Detailed Description of the Figures

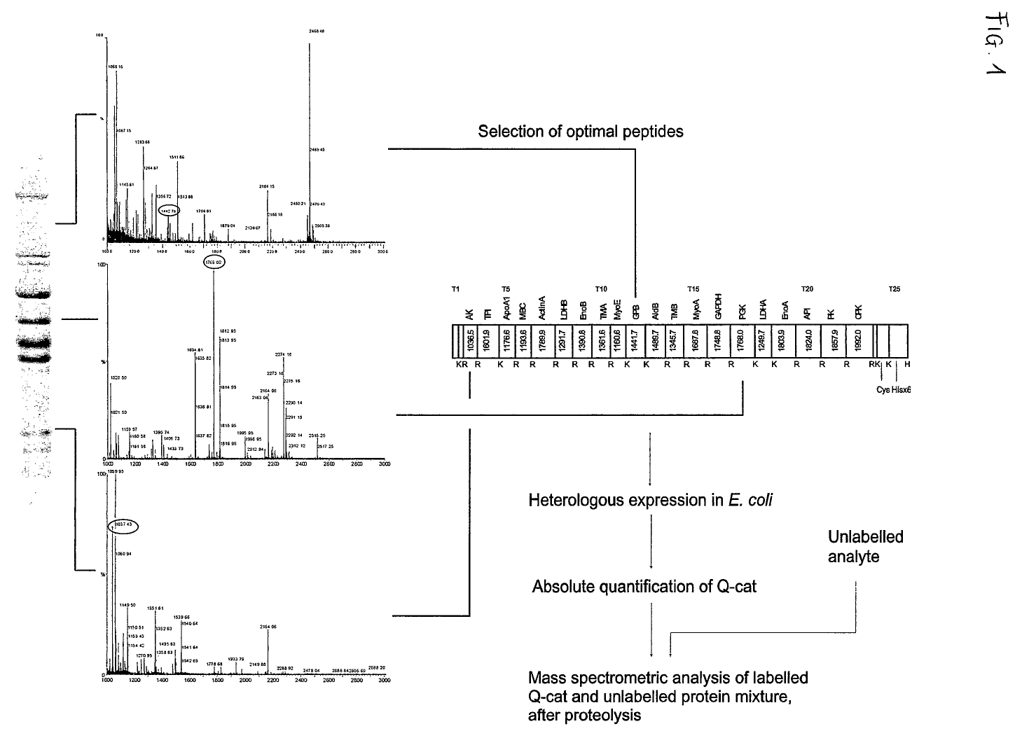

FIG. 1. Examples of Q-Peptides Selected as Signature Peptides.

For a series of proteins, peptides were selected (using multiple criteria) from proteins identified as the abundant proteins in a soluble fraction of chicken skeletal muscle, and assembled into an artificial protein, or Q-cat. Left side: Coomassie blue stained, SDS-PAGE analysis of soluble chicken muscle proteins. Center: MALDI-ToF spectrum of tryptic digestion of gel slices corresponding to selected protein bands. The peptide ion labelled with an oval represents the peptide chosen for inclusion in the Q-cat protein, Right side: mass and position of the indicated signature peptides within the designed Q-cat protein. Details of the peptides are in Table 1.

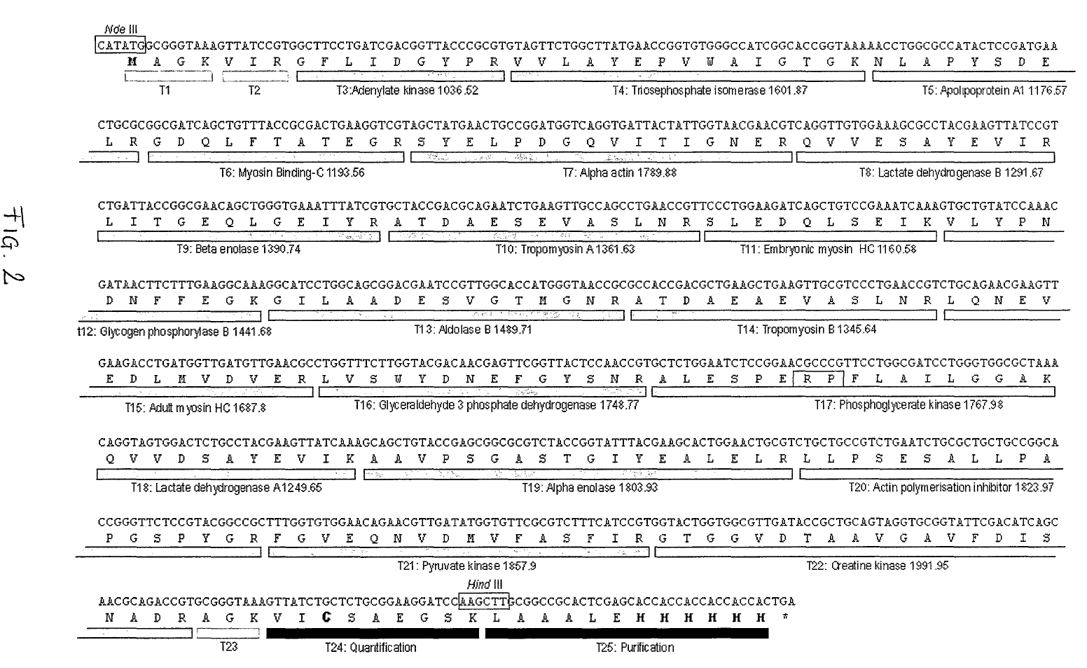

FIG. 2. The DNA Sequence, Translated Protein Sequence and Features of the QCAT.

The DNA sequence of the synthetic gene, with relevant cloning sites, is shown on the top line and the derived amino acid sequence is shown below. The grey blocked areas indicate the extent of the tryptic peptides, with the donor chicken proteins, tryptic peptide assignment (T1-T25) and the peptide mass (in Da) indicated. A non-cleavable Arg-Pro tryptic site within phosphoglycerate kinase (boxed) is included to confirm the non-digestibility of this site. Peptides (white boxes) encode the initiator methione, N-terminal sacrificial sequence and spacer sequences, and are not derived from proteins of interest. The black boxes highlight the sequences carrying the unique cysteine residue for quantification and His₆ tag for purification. T1 and T2 are sacrificial peptides designed to protect the N-terminus of the first true Q-peptide (T3)

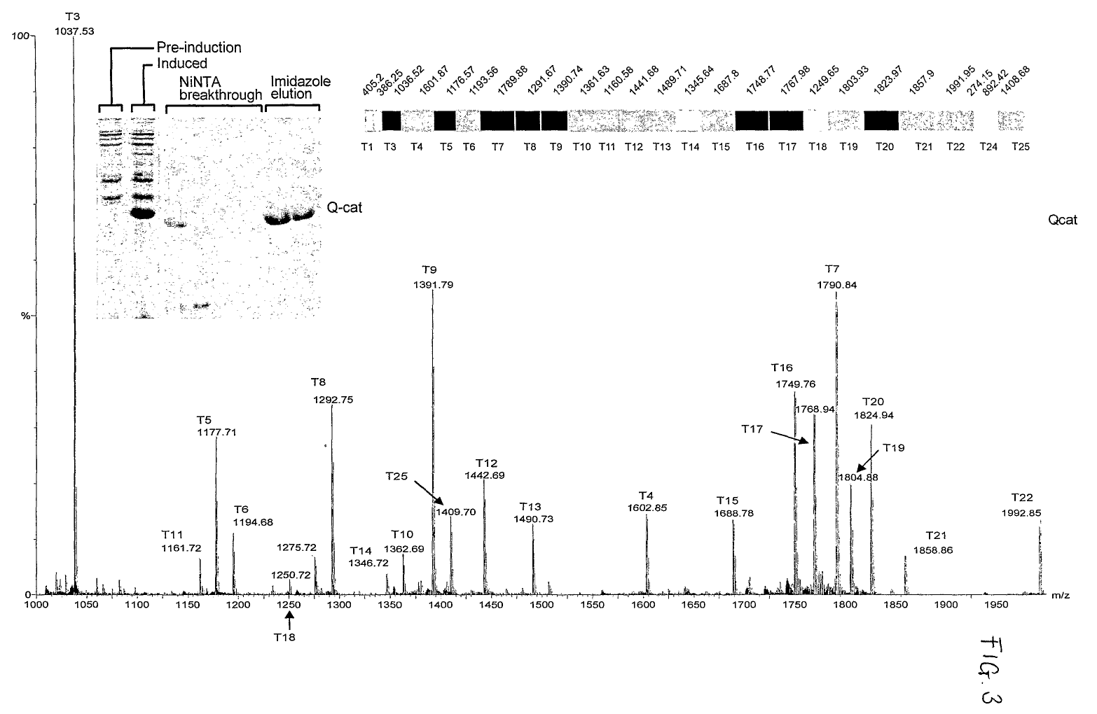

FIG. 3. Characterisation of the QCAT Protein and Q-Peptides

The pET21a/QCAT plasmid was transformed into E. coli DE3 cells and after a period of exponential growth, the expression of the QCAT was induced with IPTG. The cell lysates from pre-induced and induced cells were compared on SDS-PAGE (inset). After solubilization of the pellet, and affinity chromatography on a NiNTA column, the purified QCAT protein was homogeneous, and was digested in solution with trypsin. The peptides were analysed on MALDI-ToF mass spectrometry. The inset tryptic digestion map is shaded to indicate the relative intensities of signals corresponding to each peptide in the mass spectrum; peptides smaller than 900 Da, derived from the ‘sacrificial’ parts of the QCAT are less readily detected in this type of mass spectrometric analysis due to interfering ions.

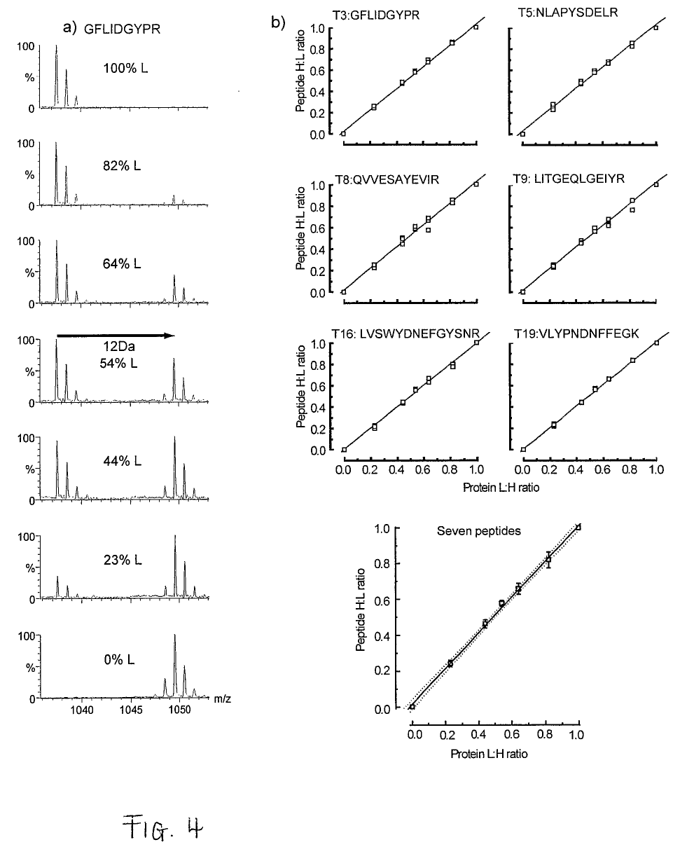

FIG. 4. Quantification Using the QCAT Protein

The QCAT protein was prepared in unlabelled form (L: “light”) and in a form uniformly labelled with ¹⁵N(H: “heavy”). The H and L QCAT proteins were separately purified, quantified and mixed in different ratios, before tryptic digestion and measurement of peptide intensities by MALDI-ToF mass spectrometry. Panel a) illustrates the mass spectrum for the Q-peptide for adenylate kinase (GFLIDGYPR, 12 nitrogen atoms). In panel b) the measured L:H ratios were plotted relative to the mixture ratio, in a triplicate series of experiments for which individual points are shown. In the bottom panel, the data for seven peptides are collated and expressed as mean±SD (n=18-21). The dotted line defines the 95% confidence limits of the fitted straight line.

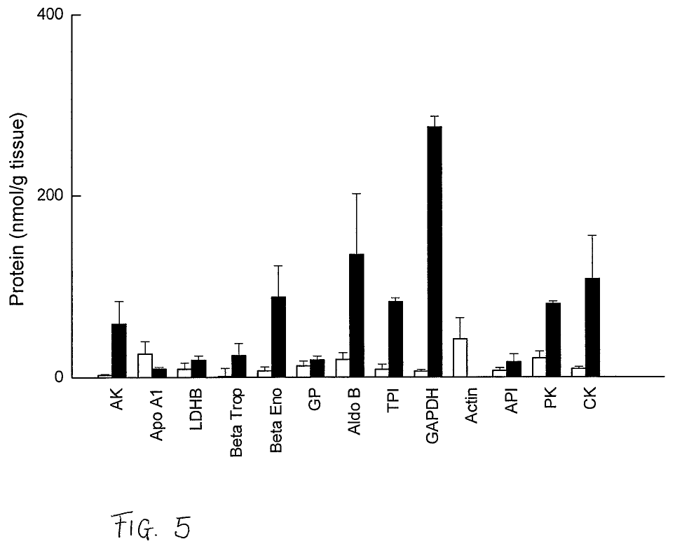

FIG. 5. Use of the QCAT for Muscle Protein Quantification.

A preparation of soluble proteins from skeletal muscle of chicks at 1d and 27d was mixed with [¹⁵N] QCAT, digested with trypsin and analysed by MALDI-ToF MS. For a subset of proteins, it was possible to determine the intensities of the endogenous and standard peptide, and from this, calculate the absolute amounts (in nmol/g tissue) of each protein. Three animals were used at each time point, error bars are SEM (n=3). Proteins were AK: adenylate kinase, ApoA1: apoliporotein A1, LDHB: lactate dehydrogenase B, Beta Trop: beta tropomyosin, Beta Eno: beta enolase, GP: glycogen phosphorylase, ALDO B: aldolase B, TPI: triose phosphate isomerase, GAPDH: glyceraldehyde 3-phosphate dehydrogenase, Actin, API: actin polymerization inhibitor, PK: pyruvate kinase and CK: creatine kinase.

### 1. Design of the Gene Encoding the Q-Protein Concatamer

One of the inventors' major interests is in proteome dynamics (Pratt, Mol Cell Proteomics 1 (2002), 579-591), and in changes in protein expression during muscle development (Doherty, Proteomics 4 (2004), 2082-2093; Doherty, Proteomics in press (2005)). A system that shows dramatic developmental changes in protein expression is the chicken pectoralis skeletal muscle from immediately post-hatching to maturity. Accordingly, for the demonstration QCAT set, the inventors chose twenty chicken proteins that had been previously identified as changing in expression level in developing skeletal muscle (Doherty, Proteomics 4 (2004), 2082-2093). A single tryptic fragment was chosen to represent each protein (a “Q-peptide”), although a peptide that can be reproducibly generated by any proteolytic or chemical fragmentation could be used, and in this example, Q-peptide selection was based on theoretical and experimental criteria. The first criterion was that the Q-peptides should lack a cysteine residue, as cysteine residue could be used for quantification of the QCAT and the absence of cysteines should avoid complex intra- and inter-molecular disulphide bond formation in the expressed protein. Secondly, the peptide chosen should be unique within the set of Q-peptides. Thirdly, the Q-peptides were chosen with masses between 1000 Da and 2000 Da, corresponding to the region in MALDI-ToF mass spectra where sensitivity of detection is typically high and interfering signals are low. Finally, an operational criterion was added, inasmuch as the inventors selected peptides that were already demonstrated to give a strong signal on MALDI-ToF mass spectrometry 75% (15 out of 20) of which were Arg-terminated tryptic peptides—the propensity of such peptides to give stronger signals on MALDI-ToF mass spectrometry is well documented (Brancia, Electrophoresis 22 (2001), 552-559). A final, less important criterion was that the Q-peptides should contain at least one instance of an abundant and chemically refractory amino acid such as leucine or valine, as this would facilitate metabolic labeling with amino acids for the preparation of stable isotope labeled Q-peptides. The peptides are summarized in Table 1:

TABLE 1
Peptides selected for Q-cat protein

| | | | |
|---|---|---|---|
| Peptide | | N | |
| mass (Da) | Sequence | atoms | Parent protein |

| | | | | |
|---|---|---|---|---|
| T1 | 405.2 | MAGK | 5 | Construct, sacrificial |
| T2 | 386.25 | VIR | 6 | Construct, sacrificial |
| T3 | 1036.52 | GFLIDGYPR | 12 | Adenylate kinase |
| T4 | 1601.87 | VVLAYEPVWAIGTGK | 16 | Triose phosphate isome- |
| | | | | rase |
| T5 | 1176.57 | NLAPYSDELR | 14 | Apolipoprotein A1 |
| T6 | 1193.56 | GDQLFTATEGR | 15 | Myosin binding protein C |
| T7 | 1789.88 | SYELPDGQVITIGNER | 21 | Alpha actin |
| T8 | 1291.67 | QVVESAYEVIR | 15 | Lactate dehydrogenase B |
| T9 | 1390.74 | LITGEQLGEIYR | 16 | Beta enolase |
| T10 | 1361.63 | ATDAESEVASLNR | 17 | Alpha tropomyosin |
| T11 | 1160.58 | SLEDQLSEIK | 12 | Myosin heavy chain (em- |
| | | | | bryonic) |
| T12 | 1441.68 | VLYPNDNFFEGK | 15 | Glycogen phosphorylase |
| T13 | 1489.71 | GILAADESVGTMGNR | 19 | Aldolase B |
| T14 | 1345.64 | ATDAEAEVASLNR | 17 | Beta tropomyosin |
| T15 | 1687.8 | LQNEVEDLMVDVER | 19 | Myosin heavy chain |
| | | | | (adult) |
| T16 | 1748.77 | LVSWYDNEFGYSNR | 19 | Glyceraldehyde 3- |
| | | | | phosphate dehydro- |
| | | | | genase |
| T17 | 1767.98 | ALESPERPFLAILGGAK | 21 | Phosphoglycerate kinase |
| T18 | 1249.65 | QVVDSAYEVIK | 13 | Lactate dehydrogenase A |
| T19 | 1803.93 | AAVPSGASTGIYEALELR | 21 | Alpha enolase |
| T20 | 1823.97 | LLPSESALLPAPGSPYGR | 21 | Actin polymerization in- |
| | | | | hibitor |
| T21 | 1857.9 | FGVEQNVDMVFASFIR | 25 | Pyruvate kinase |
| T22 | 1991.95 | GTGGVDTAAVGAVFDISN | 25 | Creatine kinase |
| | | ADR | | |
| T23 | 274.15 | AGK | 4 | Construct, sacrificial |
| T24 | 892.42 | VICSAEGSK | 10 | Construct, quantification |
| T25 | 1408.68 | LAAALEHHHHHH | 24 | Construct, purification tag |

Once the candidate set was nominated, the Q-peptides were assembled and a gene was constructed, which encoded the assembled Q-peptides using codons for maximal expression in E. coli. At the C-terminus an extension was added to provide a cysteine residue and a His tag purification motif (the latter provided in this case by the vector pET21a). An additional series of amino acids was appended to the N-terminus to provide an initiator methionine residue and a sacrificial peptide, which when cleaved would expose a true Q-peptide (FIG. 1). This avoided complications due to N-formylation or removal of methionine from the N-terminus of QCAT. The transcript encoded by the initial QCAT gene was then analyzed in silico for features such as hairpin loops that might compromise translation. If such a feature was noted, the order of the Q-peptides was swapped until an acceptable mRNA structure was obtained—the sequence of Q-peptides within a QCAT is not relevant to their use as quantification standards and the order is thus amenable to such manipulation. The gene was constructed from a series of overlapping oligonucleotides and confirmed by DNA sequencing.

### 2. Expression of the QCAT

The QCAT gene was constructed with restriction sites, such that it could be inserted into a range of expression vectors (FIG. 2). In this instance, the gene (confirmed by DNA sequencing) was inserted into pET21a at the NdeI and HindIII sites and was expressed initially in E. coli (NovaBlue (DE3)) grown in rich medium. After induction by IPTG, SDS-PAGE analysis confirmed high-level expression of a protein of the expected mass (˜35 kDa). This protein was present in the insoluble fraction of sonicated cells, and was presumed to be the QCAT protein present in inclusion bodies. From this preparation we purified the QCAT protein by affinity chromatography using Ni-NTA resin, which resulted in a homogeneous preparation (FIG. 2, inset). The intact average mass of this protein, measured by ESI-MS was 33036±2 Da (data not shown), compared to the predicted mass of 33167 Da for the QCAT protein, a difference of 131 Da which is exactly consistent with loss of the methionine residue from the N-terminus. The approx. 35 kDa gel band was subjected to in-gel digestion with trypsin and analysed by MALDI-ToF MS. All predicted QCAT peptides were readily observed in the MALDI-ToF mass spectrum, although the N and C-terminal sacrificial peptidic material yielded, by design, fragments that were too small to be seen in the MALDI-ToF mass spectrum (FIG. 3). Although the peptides were chosen to yield good signals on MALDI-ToF, some peptides were markedly less intense than others. These included all of the lysine-terminated peptides, with the exception of T17, which included a non-cleavable Arg-Pro site, which although lysine terminated still yielded a strong signal on MALDI-ToF mass spectrometry. This was particularly evident in the isoform-specific Q-peptides T8 and T18, derived from lactate dehydrogenases A and B (QWESAYEVIR and QWDSAYEVIK respectively)—the lysine terminated peptide was less than 10% of the intensity of the arginine-terminated peptide, which suggests that either Q-peptides should be predominantly drawn from arginine terminated peptides, or alternatively, that a step such as guanidination should be used to convert lysine residues to homoarginine residues, enhancing the propensity to give strong signals (Brancia, Electrophoresis 22 (2001), 552-559). The QCAT was digested by trypsin very effectively and there was no evidence for partial proteolytic products of the Q-peptides, which would of course compromise the quantification step. The inventors then expressed the protein in minimal medium containing ¹⁵NH₄Cl as sole nitrogen source. When digested with trypsin, the resultant MALDI-ToF mass spectrum was of high quality, and all Q-peptides were detectable at the appropriate mass shift corresponding to the number of nitrogen atoms in the peptide (data not shown).

The unlabelled and ¹⁵N-labelled QCAT proteins were then mixed in different ratios, and digested with trypsin before the resultant limit peptides were analysed by MALDI-ToF mass spectrometry. The heavy and light variants of the peptides were readily discerned (FIG. 3a) and their intensities measured for a series of peptides (FIG. 3b). In all instances, the data were of high quality, and the relationship between proportion of material and the heavy:light ratios was linear, with a slope of one (mean±SD (n=6)=1.008±0.008), and very high correlation coefficients (r² greater than 0.99 in all instances). The combined data (FIG. 3c) expresses the data for seven peptides; the close boundaries defined by the 95% confidence limits indicate the quality of the quantification.

### Summary of Results

The inventors have applied this particular QCAT in the analysis of protein expression in chick skeletal muscle, at 1 d and 27 d post-hatching (FIG. 4). Twelve proteins present in this preparation were also represented in the QCAT. MALDI-ToF data of the tryptic peptides was readily acquired, and the changes in protein levels that occur over the first three to four weeks post-hatching were determined.

Because the proteins were absolutely quantified, the inventors were able to express the proteins as nmol per g wet weight of tissue. The variance of the triplicate analyses was small; the inventors attribute this variance to biological rather than analytical variation. The inventors have previously measured the levels of seven of these proteins by 2D gel electrophoresis and densitometry, and the correlation between the quantification using both methods was 0.82 (r², p>0.001). Recognizing that the two methods assess different representations of the proteome, such as charge-variant isoforms or total protein complement, and that the densitometirc method is inevitably imprecise, the correlation is good.

The inventors have demonstrated the feasibility of the QCAT approach for generation of a concatenated set of Q-peptides. The QCAT, designed using both theoretical and experimental considerations, was expressed at high levels, even when grown on minimal medium, and the product was successfully purified. Because the QCAT is a completely artificial construct, the inventors did not anticipate that it would fold into any recognizable three-dimensional structure, and as expected, the protein aggregated into inclusion bodies. This is an advantage as subsequent purification is simpler, only requiring resolubilization of the pellet in strong chaotropes prior to affinity purification. Further, the lack of higher order structure of the QCAT would ensure that the QCAT was digested at least as quickly as the target proteins to be quantified.

### Use of the Artificial Protein and the QCAT Peptides

There are two ways in which the concatamers will be used. First, in the direct Q-peptide method, the concatamers are used as an internal standard. The stable-isotope labelled concatamer can be directly added to a sample or cell preparation before the proteolysis step. Alternatively, for some cell systems, the concatamer quantification can be used to achieve absolute quantification of a reference strain grown under carefully defined conditions—the indirect Q-peptide approach. This reference strain can then be used, in stable isotope labelled form, as an absolute quantification standard for all future proteomics quantification studies using that organism. Once a reference strain is accurately quantified, any peptide can be used to report on a protein, rather than the restricted set used as Q-peptides and this is clearly a very attractive proposition. This extends the generality of different proteomic strategies, and creates a new niche for tagging methods such as ICAT (Gygi, J Proteome Res 1 (2002), 47-54) and ITRAQ (Ross, Molecular and Cellular Proteomics in press (2004)) in a comparative proteomics analysis of an unknown against a fully quantified strain.

However, the inventors recognize that there are many instances where this approach is not appropriate, and where a stable isotope labelled concatamer itself (the direct Q-peptide approach) will be the appropriate standard. This is particularly apposite in proteomics studies using biological material that cannot be readily pre-labelled, for example, in animal tissues or in biomarker studies.

### Particular Advantages

The strategy the inventors advocate is superior to chemical synthesis of each individual Q-peptide, usually in a stable isotope form. Whilst chemical synthesis has been used in one-off applications, the process of peptide synthesis is not sufficiently ‘clean’ as to obviate exhaustive purification of the product. Secondly, for multiplexed assays, each peptide would need to be individually quantified before use. Finally, chemically synthesized Q-peptides are a finite resource whereas repeated expression of the QCAT gene is facile. High quality absolute quantification may be an effective route to overcome the difficulties associated with current methods for comparative proteomics, whether based on gel analysis or mass spectrometry. A series of comparative studies of particular cellular systems, each by comparison to a QCAT quantified reference would not only be individually quantified, but should be sufficiently rigorous that as the data sets grow, any pairwise comparison would be robust, transferable between individual laboratories and stable over time.

It should be possible to factor in the propensity of the peptide to ionize and generate a good signal in the mass spectrometer. At present, ion intensities are not used exhaustively in the analysis of mass spectra, although there have been some recent attempts to predict intensity using knowledge-based approaches (Krause, Anal Chem 71 (1999), 4160-4165; Gay, Proteomics 2 (2002), 1374-1391; Baumgart, Rapid Commun Mass Spectrom 18 (2004), 863-868). An additional factor that must be taken into account is the choice of precursor label. Whilst uniform labeling with [¹³C] or [¹⁵N] ensures that every peptide is comprehensively labeled, it might be preferable to select Q-peptides that each contain the same amino acid that is then used as the stable isotope labeled precursor. Since most QCAT proteins would be anticipated to be assemblies of tryptic peptides, a strategy of incorporation of [¹³C₆]-lysine and [¹³C₆]-arginine would also ensure that most Q-peptides would be singly labeled and the mass offset between heavy and light peptides would be a constant 6 Da. Further, an unlabelled QCAT could be labeled in vitro using reagents advocated for comparative proteomics, enhancing all of these technologies to absolute quantification.

Without being bound by any particular theory, the inventors believe that the number of Q-peptides that could be assembled into a single QCAT is limited by the ability to achieve high-level heterologous expression of large proteins. In the example given here, the inventors chose 20 peptides of average length 15 amino acids and average molecular weight 1.5 kDa. If 100 proteins were represented in a single QCAT, the resultant recombinant protein would be 150 kDa, which should be readily expressed. The entire yeast proteome, of approx 6000 proteins, could then be defined within approx. 60 QCAT constructs.

This ability to quantify as many as 100 proteins in a single construct invites the challenge of optimal assembly of individual Q-peptides in QCATs. Different criteria might be envisaged. First, a group of Q-peptides would allow absolute quantification of a particular subcellular fraction, or of a specific subset of proteins, for example transcription factors or protein kinases. Secondly, and perhaps more importantly, concatenation could be driven by the abundance of the target proteins in the cell. It would be difficult to quantify two different proteins with widely different expression levels in the same QCAT experiment, and it might be preferable to assemble high abundance proteins in a construct distinct from that encoding low abundance proteins.

Other applications for QCATs are readily envisaged, particularly in the broad areas of clinical biology and toxicology and diagnostics. Absolute quantification will add a new dimension to the predictive values of analyses in clinical or other biomarker monitoring systems and will absolutely define the stoichiometric ratios of individual proteins within a subcellular compartment or a multi-protein complex.

### Materials and Methods

Materials. [¹⁵N]H₄Cl (99% atom percent excess) was provided by CK Gas Products Ltd, Hampshire, UK. Most reagents, except where listed here, have been described previously (Doherty, Proteomics 4 (2004) 2082-2093; Pratt, Proteomics 2 (2002), 157-160). Chick (layer, Hi-Sex Brown) skeletal muscle proteins were pre-pared from 1 d and 27 d old chicks as a 20.000 g supernatant of a 10% (w/v) homogenate (Doherty, Proteomics 4 (2004) 2082-2093; Doherty, Proteomics 5 (2005) 5, 522-533.

QCAT gene design and construction. Q-peptides were selected for uniqueness of mass, propensity to ionise and be detectable in mass spectrometry, the presence of specific amino acid residues (for example, leucine or valine), the absence of other amino acid residues (cysteine, histidine, methionine). The peptide sequences were then randomly concatenated in silico and used to direct the design of a gene, codon-optimised for expression in E. coli. The predicted transcript was analysed for RNA secondary structure that might diminish expression, and if this was present, the order of the peptides was altered. N-and C-terminal sequences were added as sacrificial structures, protecting the assembly of true Q-peptides from exoproteolytic attack during expression. Additional peptide sequences were added to provide an initiator methionine and a C-terminal cysteine residue for quantification. The artificial gene was synthesised de novo (by Entelechon GmbH, Germany) from a series of overlapping oligonucleotides, verified by DNA sequencing and ligated into the NdeI and Hind III sites of the pET21a expression vector, to yield the QCAT plasmid, pET21a QCAT. A His₆ purification tag was provided by fusion to the vector.

QCAT gene expression and labeling with 15N. The QCAT plasmid, pET21a QCAT, was used to transform NovaBlue (DE3) (K-12 endA1, hsdR17(r_{K12}⁻m_{K12}⁺), supE44, thi-1, recA1, gyrA96, relA1, lac, F′[proA⁺B⁺, lacl^{q}Z□M15::Tn10(Tc^{R})] cells to ampicillin resistance. Cells were grown at 37° C. in Luria broth, 100 μg/ml ampicillin to an A₆₀₀ of 0.4-0.6 and IPTG added to 1 mM. Incubation continued for a further five hours when cells were pelleted by centrifugation (5000 g, 4 min., 4° C.), resuspended in 10 ml 20 mMTris/HCl buffer, pH 8.0 and lysozyme was added (100 μg/ml) for ten minutes at room temperature. Cells were then sonicated (three bursts of 30 s) on ice and centrifuged at 14000 g for 10 min. Pellets and supernatants of induced and uninduced cultures were analysed by 12.5% (w/v) SDS PAGE/Coomassie blue staining. For ¹⁵N-labelling, cells were grown in M9 minimal medium prepared using [¹⁵N]H₄Cl (20 mM), and induced and processed as above.

Purification and analysis of the QCAT protein. The pellets from sonicated cells were dissolved in 20 mM phosphate buffer (pH 7.5) containing 20 mM imidazole and 8 M urea (Buffer A) before being applied to a NiNTA column (GE Healthcare). After 10 column volumes of washing in the same buffer, the bound material was eluted with buffer A with an increased concentration of imidazole (500 mM). This material was desalted on Sephadex G25 ‘spun columns’ and the mass of the eluted protein was determined by electrospray ionisation mass spectrometry using a Waters-Micromass Q-ToF micro mass spectrometer. The mass spectra were processed using the MaxEnt I algorithm. The purified desalted protein was digested with trypsin, and the resultant peptides were mass measured using a Waters-Micromass MALDI-ToF mass spectrometer (Doherty, Proteomics 4 (2004) 2082-2093). To assess the response ratio of heavy and light variants of the QCAT, the purified ‘heavy’ and ‘light’ proteins were mixed in different ratios prior to digestion with trypsin and MALDI-ToF mass spectrometry. The intensities of the [¹⁴N]- and [¹⁵N]-peptides were measured on centroided spectra.

Use of the QCAT to quantify muscle protein expression. The supernatant fraction containing chicken soluble proteins derived from 100 mg of tissue was mixed with 290 μg of [¹⁵N] QCAT, quantified by protein assay and digested with trypsin overnight—the QCAT was digested at a higher rate than endogenous muscle proteins (results not shown). The experiment was replicated for three animals at each time point. Subsequently, the [¹⁴N]-(muscle) and [¹⁵N]-(QCAT) peptides were identified by mass, and their relative intensities measured by MALDI-TOF mass spectrometry.

### Mass Spectrometric Analysis

The analysis in this example was carried out using a MALDI-ToF Mass Spectrometer, but this method is equally applicable to all other Mass Spectrometric methods suitable for the analysis of peptides.

### REFERENCES

Baumgart, S. et al. The contributions of specific amino acid side chains to signal intensities of peptides in matrix-assisted laser desorption/ionization mass spectrometry. Rapid Commun Mass Spectrom 18, 863-868 (2004).
Brancia, F. L. et al. A combination of chemical derivatisation and improved bioinformatic tools optimises protein identification for proteomics. Electrophoresis 22, 552-559 (2001).
Doherty, M. K. et al. The proteome of chicken skeletal muscle: changes in soluble protein expression during growth in a layer strain. Proteomics 4, 2082-2093 (2004).
Doherty, M. K., Whitehead, C., McCormack, H., Gaskell, S. J. & Beynon, R. J. Proteome dynamics in complex organisms: using stable isotopes to monitor individual protein turnover rates. Proteomics 5, 522-533. (2005)
Dunkley, T. P., Watson, R., Griffin, J. L., Dupree, P. & Lilley, K. S. Localization of organelle proteins by isotope tagging (LOPIT). Mol Cell Proteomics (2004).
Gay, S., Binz, P. A., Hochstrasser, D. F. & Appel, R. D. Peptide mass fingerprinting peak intensity prediction: extracting knowledge from spectra. Proteomics 2, 1374-1391 (2002).
Gerber, S. A., Rush, J., Stemman, O., Kirschner, M. W. & Gygi, S. P. Absolute quantification of proteins and phosphoproteins from cell lysates by tandem MS. Proc Natl Acad Sci USA 100, 6940-6945 (2003).
Gygi, S. P., Rist, B., Griffin, T. J., Eng, J. & Aebersold, R. Proteome analysis of low-abundance proteins using multidimensional chromatography and isotopecoded affinity tags. J Proteome Res 1, 47-54 (2002).
Hoang, V. M. et al. Quantitative proteomics employing primary amine affinity tags. J Biomol Tech 14, 216-223 (2003).
Julka, S. & Regnier, F. Quantification in proteomics through stable isotope coding: a review. J Proteome Res 3, 350-363 (2004).
Krause, E., Wenschuh, H. & Jungblut, P. R. The dominance of arginine-containing peptides in MALDI-derived tryptic mass fingerprints of proteins. Anal Chem 71, 4160-4165 (1999).
Lopez, M. F. & Pluskal, M. G. Protein micro- and macroarrays: digitizing the proteome. J Chromatogr B Analyt Technol Biomed Life Sci 787, 19-27 (2003).
Ong, S. E. et al. Stable isotope labeling by amino acids in cell culture, SILAC, as a simple and accurate approach to expression proteomics. Mol Cell Proteomics 1, 376-386 (2002).
Pratt, J. M. et al. Stable isotope labelling in vivo as an aid to protein identification in peptide mass fingerprinting. Proteomics 2, 157-163 (2002).
Pratt J M, Petty J, Riba-Garcia I, Robertson D H, Gaskell S J, Oliver S G, Beynon R J. Dynamics of protein turnover, a missing dimension in proteomics. Mol Cell Proteomics. 2002 August; 1 (8):579-991 (2002).
Righetti, P. G., Campostrini, N., Pascali, J., Hamdan, M. & Astner, H. Quantitative proteomics: a review of different methodologies. Eur J Mass Spectrom (Chichester, Eng) 10, 335-348 (2004).
Ross, P. L. et al. Multiplexed protein quantitiation in Saccharomyces cerevisiae using amine-reactive isobaric tagging reagents. Molecular and Cellular Proteomics (in press) (2004).
Sechi, S. & Oda, Y. Quantitative proteomics using mass spectrometry. Curr Opin Chem Biol 7, 70-77 (2003).
Walter, G., Bussow, K., Lueking, A. & Glokler, J. High-throughput protein arrays: prospects for molecular diagnostics. Trends Mol Med 8, 250-253 (2002).
WO 03/102220 (Aebersold, R.)

1. Artificial protein for quantitative analysis of the proteome of a sample, cell or organism, comprising:
(a) at least two consecutive peptides linked by a cleavage sequence for separating the peptides;
(b) a singular marker on one or more peptides for determination of the absolute amount of the protein; and
(c) N-terminal and C-terminal extensions for protection of the peptides; wherein each peptide represents one single protein of the sample, cell or organism and each peptide is in a defined stoichiometry.

2. Artificial protein of claim 1, wherein the protein comprises 10-200 peptides.

3. Artificial protein of claim 1, wherein the protein comprises 10-100 peptides.

4. Artificial protein of claim 1, wherein the protein comprises 20-60 peptides.

5. Artificial protein of claim 1, wherein the protein comprises 60 peptides.

6. Artificial protein of claim 1, wherein the cleavage sequence is cleaved by a protease.

7. Artificial protein of claim 6, wherein the cleavage sequence is cleaved by trypsin.

8. Artificial protein of claim 1, wherein the singular marker is a cysteine residue.

9. Artificial protein of claim 1, wherein one or more peptides are repeated identically one or more times, in order to achieve a particular stoichiometry between all peptide species.

10. Artificial protein of claim 1, wherein the protein comprises an affinity tag for purification of the protein.

11. Artificial protein of claim 1, wherein the protein is labelled by an isotope.

12. Artificial protein of claim 1, wherein each peptide comprises between about 3 and 40 amino acids.

13. Artificial protein of claim 12, wherein each peptide comprises about 15 amino acids.

14. Artificial protein of claim 1, wherein the peptides represent different conformational, metabolic or modification states of the protein.

15. Artificial protein of claim 1, wherein the peptides have a defined molecular weight distribution and quantitative ratios.

16. A collection of peptides as defined in step (b) of claim 1, which covers the complete proteome of an organism to allow the rapid quantification of the proteome of such an organism.

17. Vector comprising a nucleic acid encoding the artificial protein of claim 1.

18. Kit comprising the vector of claim 17.

19. A method for quantitative analysis of the proteome of a sample, cell or organism, comprising the steps of:
(a) quantifying the amount of the protein or one peptide containing the singular marker in an absolute manner;
(b) generating a preparation of the proteins to be quantified;
(c) mixing the products of steps (a) and (b);
(d) completely cleaving the artificial protein of claim 1 and the proteins to be quantified in step (b) at the cleavage sequence;
(e) determining the quantitative amount of peptides;
(f) calculating the absolute amount of peptides,
wherein the artificial protein and/or the peptides are isotopically labelled.
